(19) United States
(12) Patent Application Publication
(10) Pub. No.: US 2006/0135553 A1
(43) Pub. Date: Jun. 22, 2006

(54) Imidazole derivatives

(76) Inventors: David A. Campbell, San Diego, CA (US); Juan M. Betancort, San Diego, CA (US)

Correspondence Address: FOLEY & LARDNER LLP, 150 EAST GILMAN STREET, P.O. BOX 1497, MADISON, WI, 53701-1497, US

(21) Appl. No.: 11/262,394

(22) Filed: Oct. 28, 2005

Related U.S. Application Data
(60) Provisional application No. 60/623,421, filed on Oct. 28, 2004.

(51) Int. Cl. A61K 31/4745; A61K 31/444; C07D 471/02; C07D 403/14

(52) U.S. Cl. 514/303; 514/332; 546/117; 546/256

(57) ABSTRACT

Certain imidazole-containing compounds are useful for treating and/or preventing of various disease conditions, by way of methodology for modulating tyrosine kinases and angiogenesis. Illustrative of such conditions are inflammatory diseases and diseases characterized by abnormal cellular proliferation.

### CROSS-REFERENCE TO RELATED PATENT APPLICATIONS

This application claims priority under 35 U.S.C. § 119 to U.S. Provisional Application No. 60/623,421, filed Oct. 28, 2004, which is hereby incorporated by reference in its entirety and for all purposes.

### FIELD OF THE INVENTION

The present invention relates to pharmaceutical compounds useful in the treatment of human diseases. In particular, the present invention pertains to imidazole derivatives, and methods of preparing and using the same in the treatment of cancer and inflammatory diseases.

### BACKGROUND OF THE INVENTION

The information provided and publications cited in this description are solely to assist the understanding of the reader. Mention of this information and these publications is not an admission that either the information or any publication represents prior art to the present invention.

Tyrosine kinases are a class of enzymes that catalyze the transfer of the terminal phosphate of adenosine triphosphate to tyrosine residues in protein substrates. Tyrosine kinases are implicated in signal transduction for a number of cell functions via substrate phosphorylation. Though the exact mechanism of signal transduction is still unclear, tyrosine kinases have been shown to be important contributing factors in cell proliferation, carcinogenesis and cell differentiation.

Tyrosine kinases can be of the receptor type or non-receptor type. Generally, receptor type tyrosine kinases have an extracellular, a transmembrane, and an intracellular portion, whereas non-receptor type tyrosine kinases are wholly intracellular.

A large number of transmembrane receptors, some with diverse biological activity, are receptor-type tyrosine kinases. Currently, approximately twenty different subfamilies of receptor-type tyrosine kinases have been identified. Exemplary tyrosine kinase subfamilies include the HER subfamily, which comprises receptors EGFR, HER2, HER3, and HER4. Ligands for the HER receptor subfamily include epithelial growth factor, TGF-α, amphiregulin, HB-EGF, betacellulin, and heregulin. Other exemplary tyrosine kinase subfamilies include the insulin subfamily, which comprises the INS-R, IGF-R, and IR-R receptors; the PDGF subfamily, which comprises the PDGF-α, PDGF-β, CSFIR, c-kit, and FLK-II receptors; the FLK subfamily, which comprises the kinase insert domain receptor (KDR), fetal liver kinase-1 (FLK-1), fetal liver kinase-4 (FLK-4), and fms-like tyrosine kinase-1 (flt-1) receptors. For a more detailed discussion of the receptor-type tyrosine kinases, see, for example, Plowman et al. (“Receptor Tyrosine Kinases As Targets For Drug Intervention,” Drug News & Perspectives, Vol. 7(6): 334-339 (1994)).

Non-receptor tyrosine kinases also contain numerous protein subfamilies of which include Src, Frk, Btk, Csk, Abl, Zap70, Fes/Fps, Fak, Jak, Ack, and LiMK. Members of the Src subfamily, which is the largest non-receptor tyrosine kinase subfamily, include Src, Yes, Fyn, Lyn, Lck, Blk, Hck, Fgr, and Yrk. For a more detailed discussion of the non-receptor type of tyrosine kinases, see Bolen et al. (“Nonreceptor tyrosine protein kinases,” Oncogene, Vol. 8(8): 2025-2031 (1993)).

Both the receptor-type and non-receptor type tyrosine kinases have been implicated in cellular signaling pathways leading to numerous pathogenic conditions, including cancer, psoriasis, and hyperimmune responses.

Several receptor-type tyrosine kinases, and the growth factors that bind thereto, have been suggested to play a role in angiogenesis, and some may promote angiogenesis indirectly (Mustonen et al., J. Cell Biol., Vol. 129: 895-898 (1995)). One such implicated receptor-type tyrosine kinase is fetal liver kinase 1 (FLK-1). The human analog of FLK-1 is the kinase insert domain-containing receptor (KDR), also referred to as vascular endothelial cell growth factor receptor 2 (VEGFR-2) because it binds VEGF with high affinity. The VEGF and KDR ligand-receptor interaction play a pivotal role in mediation of vasculogenesis and angiogenesis.

VEGF is composed of a family of ligands. VEGF binds various tyrosine kinase receptors including KDR and flt-1, also referred to as vascular endothelial cell growth factor receptor 1 (VEGFR-1). Cell culture and gene knockout experiments indicate that each receptor contributes to different aspects of angiogenesis. For instance, KDR mediates the mitogenic function of VEGF whereas flt-1 appears to modulate functions associated with cellular adhesion. Inhibiting KDR thus modulates the level of mitogenic VEGF activity.

The use of VEGF receptor antagonists to effect anti-angiogenesis on tumor growth has been reported (Kim et al., Nature 362, pp. 841-844, 1993). For instance, treatment of solid tumors with tyrosine kinase inhibitors have been reported (Herbst et al., J Clin. Oncol., Vol. 20(18): 3815-25 (2002) and Slichenmyer et al., Semin Oncol., 5 Suppl 16: 80-5 (2001)). Exemplary solid tumors include histiocytic lymphoma, cancers of the brain, genitourinary tract, lymphatic system, stomach, larynx, and lung (including lung adenocarcinoma and small cell lung cancer). Additional examples of cancers in which treatment with tyrosine kinase inhibitors may be effective include those cancers associated with over-expression or activation of Raf-activating oncogenes (e.g., K-ras or erb-B), such as pancreatic and breast carcinoma.

The angiogenic activity of VEGF is not limited to tumors. For example, VEGF accounts for most of the angiogenic activity produced in or near the retina in diabetic retinopathy. Vascular growth in the retina leads to visual degeneration culminating in blindness. Levels of ocular VEGF mRNA and protein are elevated, in part, by retinal vein occlusion in primates and decreased pO₂ levels in mice. Inhibition of retinal neovascularization in primate and rodent models with intraocular injections of anti-VEGF monoclonal antibodies or VEGF receptor immunofusions has been reported (Csaky et al., “Anti-vascular endothelial growth factor therapy for neovascular age-related macular degeneration: promises and pitfalls,” Ophthalmology, Vol. 110(5): 879-81 (2003)). Thus, modulation of VEGF activity and expression presents a viable means to treat diabetic retinopathy.

### SUMMARY OF THE INVENTION

The present invention provides imidazole-containing compounds that may be useful in the treatment of various diseases. In a preferred embodiment, compounds are employed to inhibit, regulate, and/or modulate tyrosine kinase activity, including the signal transduction of tyrosine kinases. Accordingly, compounds presented herein may be administered to treat and/or prevent indications such as neoangiogenesis, cancer, atherosclerosis, diabetic retinopathy, inflammatory diseases, and the like.

An aspect of the invention is drawn to compounds corresponding to Formula (I): 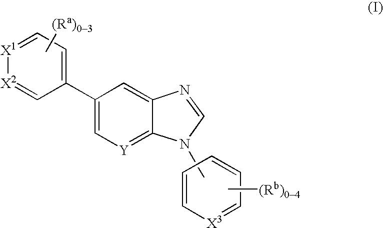
wherein:
each of X¹ and X² is independently CH, CR^{a}, or N, provided that at least one of X¹ or X² is CR^{a};
X³ is CH, CR^{b}, or N;
Y is CH or N;
R^{a} at each occurrence is independently alkenyl, alkynyl, OR¹, C(O)NR²R³, C(O)NR²(C₁-C₆ alkylene-R¹), C(O)OR², O—(C₁-C₆ alkylene)-C(O)NR²R³, O—(C₁-C₆ alkylene)-C(O)NR²(C₁-C₆ alkylene-R¹), SR², SO₂R², SO₂NR²R³, NR²SO₂R³, SO₃R², O—(C₁-C₆ alkylene)-NR²C(O)R³, O—(C₁-C₆ alkylene)-NR²C(O)(C₁-C₆ alkylene-R¹), NR²—(C₁-C₆ alkylene)-C(O)NR²R³; or NR²—(C₁-C₆ alkylene)-C(O)NR²(C₁-C₆ alkylene-R¹);
R^{b} is optional, and when present at each occurrence is independently R², halogen, alkyl, alkenyl, alkynyl, alkoxy, C₁-C₆ alkylene-R¹, NH(C₁-C₆ alkylene-R¹), OR¹, OR², NR²R³, NO₂, C(O)NR²R³, C(O)OR², O—(C₁-C₆ alkylene)-C(O)NR²R³, SR², SO₂R², SO₂NR²R³, NR²SO₂R³, SO₃R², O—(C₁-C₆ alkylene)-NR²C(O)R³, or N—(C₁-C₆ alkylene)-C(O)NR²R³;
R¹ at each occurrence is independently selected from aryl, heteroaryl, or heterocyclyl; and
R² and R³ are at each occurrence independently selected from hydrogen, alkyl, alkenyl, alkynyl, alkoxy, aryl, heteroaryl, cycloalkyl, cycloalkylalkyl, heterocyclyl, or heterocyclylalkyl; or R² and R³, are taken together, to form a cycloalkyl or heterocyclyl.

Preferred embodiments of Formula (I) include compounds corresponding to Formula (II):

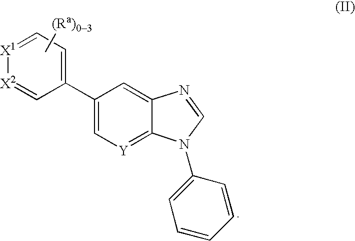

Additionally preferred embodiments of Formula (I) include compounds corresponding to Formula (III):

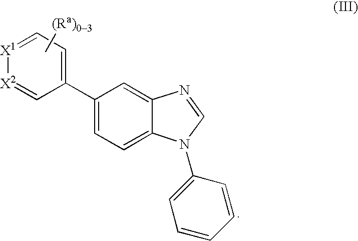

In some embodiments of compounds of the invention, X² is CH and X¹ is CR^{a}, such as when R^{a} is SO₂(alkyl), C(O)NH₂, C(O)NH—(C₁-C₆ alkylene)-heterocyclyl, SO₂NR²R³, or O—(C₁-C₆ alkylene)-C(O)NH—(C₁-C₆ alkylene)-heterocyclyl. In some embodiments, X¹ is CH and X² is CR^{a}. In some such embodiments, R^{a} is C(O)NR²R³ or SO₂R². In other such embodiments, R^{a} is C(O)NH—(C₁-C₆ alkylene)-heterocyclyl. Exemplary compounds of the invention are set forth in the Examples, particularly in Table 2.

Embodiments include compositions comprising compounds and a pharmaceutically acceptable carrier; pharmaceutical compositions comprising compounds and a pharmaceutically acceptable carrier; and kits comprising a vessel containing compounds.

An aspect of the invention is drawn to methods of modulating tyrosine kinase activity comprising administering an effective amount of a compound presented herein, whereby activity of said tyrosine kinase is altered.

Another aspect of the invention is drawn to methods of modulating angiogenesis comprising administering an effective amount of a compound presented herein, whereby said compound modulates a tyrosine kinase involved in angiogenesis.

Yet another aspect of the invention is drawn to methods of treating a disease characterized by abnormal cellular proliferation or treating an inflammatory disease comprising administering a therapeutically effective amount of a compound presented herein to a patient in need thereof. Representative cancers which may be treated and/or prevented using compounds include cancers of the brain, genitourinary tract, lymphatic system, stomach, larynx, and lung. Representative inflammatory disease which may be treated and/or prevented using compounds include rheumatoid arthritis, psoriasis, contact dermatitis, and delayed hypersensitivity reactions.

The present invention also provides methods for preparing compounds by combining a compound corresponding to formula (IV) 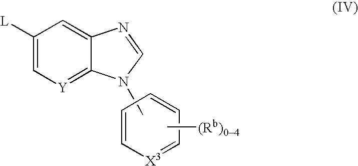
with a compound corresponding to Formula (V) in the presence of a palladium catalyst, wherein the compound of Formula (V) corresponds to: 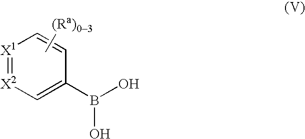
wherein:
   L is a leaving group;
   each of X¹ and X² is independently CH, CR^{a}, or N, provided that at least one of X¹ or X² is CR^{a};
   X³ is CH, CR^{b}, or N;
   Y is CH or N;
   R^{a} at each occurrence is independently alkenyl, alkynyl, OR¹, C(O)NR²R³, C(O)NR²(C₁-C₆ alkylene-R¹), C(O)OR², O—(C₁-C₆ alkylene)-C(O)NR²R³, O—(C₁-C₆ alkylene)-C(O)NR²(C₁-C₆ alkylene-R¹), SR², SO₂R², SO₂NR²R³, NR²SO₂R³, SO₃R², O—(C₁-C₆ alkylene)-NR²C(O)R³, O—(C₁-C₆ alkylene)-NR²C(O)(C₁-C₆ alkylene-R¹), NR²—(C₁-C₆ alkylene)-C(O)NR²R³; or NR²—(C₁-C₆ alkylene)-C(O)NR²(C₁-C₆ alkylene-R¹);
   R^{b} is optional, and when present at each occurrence is independently R², halogen, alkyl, alkenyl, alkynyl, alkoxy, C₁-C₆ alkylene-R¹, NH(C₁-C₆ alkylene-R¹), OR¹, OR², NR²R³, NO₂, C(O)NR²R³, C(O)OR², O—(C₁-C₆ alkylene)-C(O)NR²R³, SR², SO₂R², SO₂NR²R³, NR²SO₂R³, SO₃R², O—(C₁-C₆ alkylene)-NR C(O)R³, or N—(C₁-C₆ alkylene)-C(O)NR²R³;
   R¹ at each occurrence is independently selected from aryl, heteroaryl, or heterocyclyl; and
   R² and R³ are at each occurrence independently selected from hydrogen, alkyl, alkenyl, alkynyl, alkoxy, aryl, heteroaryl, cycloalkyl, cycloalkylalkyl, heterocyclyl, or heterocyclylalkyl; or R² and R³, are taken together, to form a cycloalkyl or heterocyclyl.

In some embodiments of methods for preparing compounds of the invention, L is Br, I, or triflate. In other embodiments, compounds of formul (IV) have the structure (1B):

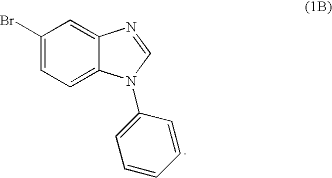

In some embodiments, methods for preparing compounds of the invention include methods involving compounds of Formula (V) where X² is CH and X¹ is CR^{a}. In some such embodiments, R^{a} is SO₂(alkyl), C(O)NR²R³, SO₂NR²R³, or C(O)OR². For example, compounds of Formula V for use in the present methods include but are not limited to:

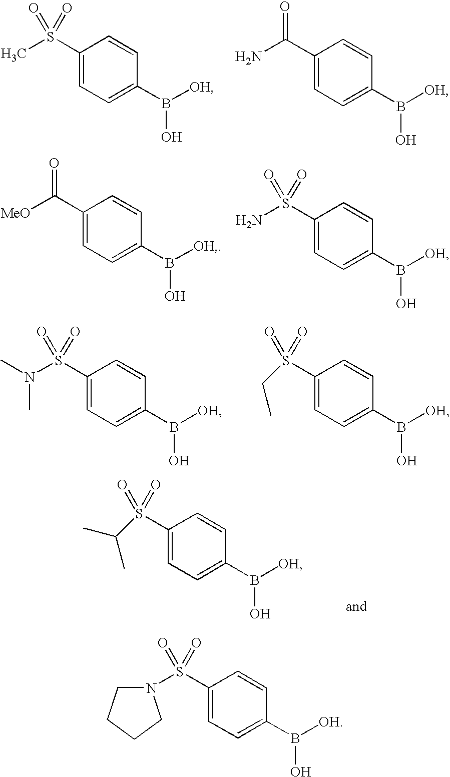

Other embodiments further include preparative methods involving compounds of Formula (V) where X¹ is CH and X² is CR^{a}. In some such embodiments, R^{a} is SO₂(alkyl), C(O)NR²R³, SO₂NR²R³, or C(O)OR². Suitable compounds of Formula V include, e.g.,

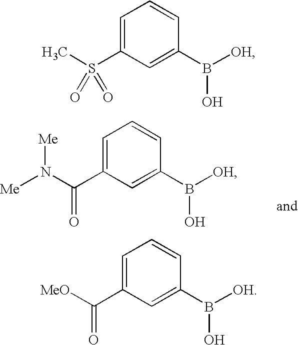

In some methods for preparing compounds, the palladium catalyst is Pd(Ph₃P)₄, PdCl₂(dppf).CH₂Cl₂, or PdCl₂(Ph₃P)₂.

### DETAILED DESCRIPTION OF THE INVENTION

I. Compounds

In this description, the reference to a certain element, such as hydrogen or H, connotes all isotopes of that element. For instance, if a group is defined to include hydrogen or H, it also can include deuterium and/or tritium.

Compounds of the present invention may have asymmetric centers and may occur, except when specifically noted, as mixtures of stereoisomers or as individual diastereomers, or enantiomers, with all isomeric forms being included in the present invention. Compounds of the present invention embrace all conformational isomers. Compounds of the present invention may also exist in one or more tautomeric forms, including both single tautomers and mixtures of tautomers.

The phrase “substituted” refers to an atom or group of atoms that has been replaced with another substituent. The phrase “substituted” includes any level of substitution, e.g. mono-, di-, tri-, tetra-, or penta-substitution, where such substitution is chemically permissible. Substitutions can occur at any chemically accessible position and on any atom, such as substitution(s) on carbons or any heteroatom. For example, substituted compounds are those where one or more bonds to a hydrogen or carbon atom(s) contained therein are replaced by a bond to non-hydrogen and/or non-carbon atom(s). Substitutions can include, but are not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines (NH₂), amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups.

The phrase “hydrocarbyl” refers to any organic radical having a directly attachable carbon atom to any molecule presented herein. The phrase “substituted hydrocarbyl” refers to a hydrocarbyl group that is substituted according to the definition provided above. Hydrocarbyl groups include saturated and unsaturated hydrocarbons, straight and branched chain aliphatic hydrocarbons, cyclic hydrocarbons, and aromatic hydrocarbons.

The phrase “alkyl” refers to saturated hydrocarbyl chains comprising from 1 to 20 carbon atoms. The phrase “alkyl” includes straight chain alkyl groups, such as methyl, ethyl, propyl, butyl, pentyl, hexyl, heptyl, octyl, nonyl, decyl, undecyl, dodecyl, and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, and —CH₂CH(CH₃)₂. Thus, alkyl groups include primary alkyl groups, secondary alkyl groups, and tertiary alkyl groups. Preferred alkyl groups include alkyl groups having from 1 to 10 or 1 to 6 carbon atoms, such as those having from 1 to 3 carbon atoms, e.g., methyl, ethyl, and propyl. Alkyl groups embrace “substituted alkyl” group, wherein an alkyl group is substituted according to the definition provided above.

The phrase “alkylene” refers to a divalent alkyl group, in which the alkyl is as defined above.

The phrase “alkenyl” refers to hydrocarbyl chains comprising from 2 to 20 carbon atoms and comprising at least one carbon-carbon double bond (—C═C—). The phrase “alkenyl” includes straight chain alkenyl groups, as well as branched chain isomers of straight chain alkenyl groups. Preferably, alkenyl groups comprise from 1 to 8 double bond(s). The phrase “substituted alkenyl” refers to an alkenyl group that is substituted according to the definition provided above.

The phrase “alkynyl” refers to hydrocarbyl chains comprising from 2 to 20 carbon atoms and comprising at least one carbon-carbon triple bond (—C≡C—). The phrase “alkynyl” includes straight chain alkynyl groups, as well as branched chain isomers of straight chain alkynyl groups. In some embodiments, alkynyl groups comprise from 1 to 4 triple bond(s). The phrase “substituted alkynyl” refers to an alkynyl group that is substituted according to the definition provided above.

The phrase “alkoxy” refers to an oxygen-containing alkyl group, as defined above.

The phrase “halogen” refers to a substituent selected from F, Cl, I, or Br. A preferred halogen is F.

The phrase “aryl” refers to aromatic radicals that comprise from 3 to 20 carbon atoms, and, in some embodiments, from 6 to 12 carbon atoms. Aryl groups include monocyclic, bicyclic, or polycyclic aromatic rings, such as, but not limited to, phenyl, biphenyl, anthracenyl, and naphthenyl. Aryl groups embrace “substituted aryl group”, which refers to an aryl group that is substituted according to the definition provided above. For example, substituted aryl groups may be bonded to one or more carbon atom(s), oxygen atom(s), nitrogen atom(s), and/or sulfur atom(s) and also includes aryl groups in which one or more aromatic carbons of the aryl group is bonded to a substituted and/or unsubstituted alkyl, alkenyl, or alkynyl group.

The phrase “heteroaryl” refers to a 3 to 20-membered aromatic ring comprising carbon atoms and heteroatoms, such as N, S, and O. Heteroaryl rings include but are not limited to 5- to 6-membered monocyclic rings or an 8- to 10-membered bicyclic or polycyclic ring system containing carbon atoms and heteroatoms, such as N, S, and O, wherein at least one of the rings in the bicyclic system is an aromatic ring. The heteroaryl ring may be attached at any heteroatom or carbon atom. Representative heteroaryl compounds include, for example, imidazolyl, pyridyl, pyrazinyl, pyrimidinyl, thiophenyl, thiazolyl, furanyl, pyridofuranyl, pyrimidofuranyl, pyridothienyl, pyridazothienyl, pyridooxazolyl, pyridazooxazolyl, pyrimidooxazolyl, pyridothiazolyl, pyridazothiazolyl, pyrrolyl, pyrrolinyl, imidazolyl, pyrazolyl, pyridyl, dihydropyridyl, pyrimidyl, pyrazinyl, pyridazinyl, triazolyl (e.g. 4H-1,2,4-triazolyl, 1H-1,2,3-triazolyl, and 2H-1,2,3-triazolyl), tetrazolyl, (e.g. 1H-tetrazolyl and 2H tetrazolyl), pyrrolidinyl, imidazolidinyl, piperidinyl, piperazinyl, indolyl, isoindolyl, indolinyl, indolizinyl, benzimidazolyl, quinolyl, isoquinolyl, indazolyl, benzotriazolyl, oxazolyl, isoxazolyl, oxadiazolyl (e.g. 1,2,4-oxadiazolyl, 1,3,4-oxadiazolyl, and 1,2,5-oxadiazolyl), benzoxazolyl, benzoxadiazolyl, benzoxazinyl (e.g. 2H-1,4-benzoxazinyl), thiazolyl, isothiazolyl, thiadiazolyl (e.g. 1,2,3-thiadiazolyl, 1,2,4-thiadiazolyl, 1,3,4-thiadiazolyl, and 1,2,5-thiadiazolyl). Heteroaryl groups embrace “substituted heteroaryl”, which refers to a heteroaryl group that is substituted according to the definition provided above.

The phrase “cycloalkyl” refers to an alicyclic moiety having 3 to 20 carbon atoms and comprising any chemically permissible amount of saturated or unsaturated bonds. Preferably, cycloalkyl groups comprise from 4 to 7 carbons atoms. Cycloalkyl groups include, but are not limited to, cyclopropyl, cyclobutyl, cyclopentyl, cyclohexyl, cycloheptyl, cyclooctyl, and the like. The phrase “substituted cycloalkyl” refers to a cycloalkyl group that is substituted according to the definition provided above. Substituted cycloalkyl groups can have one or more atom substituted with straight or branched chain alkyl groups and can further comprise cycloalkyl groups that are substituted with other rings including fused rings. Examples of cycloalkyl groups that are substituted with fused rings include, but are not limited to, adamantyl, norbornyl, bicyclo[2.2.2]octyl, decalinyl, tetrahydronaphthyl, and indanyl, bornyl, camphenlyl, isocamphenyl, and carenyl groups. Representative substituted cycloalkyl groups may be mono-substituted or substituted more than once, such as, but not limited to, 2,2-, 2,3-, 2,4-, 2,5-, or 2,6-disubstituted cyclohexyl groups or mono-, di- or tri-substituted norbornyl or cycloheptyl groups, which may be substituted with, for example, alkyl, alkoxy, amino, thio, or halo groups. The phrase “cycloalkylalkyl” refers to a cycloalkyl attached through an alkylene moiety. Examples of cycloalkylalkyl include without limitation cyclohexyl-CH₂—, cyclohexyl-(CH₂)₂—, and the like.

The phrases “heterocycle”, “heterocyclic” and “heterocyclyl” refer to non-aromatic cyclic hydrocarbyl compounds of which at least one ring member is a heteroatom. Heterocyclic groups include monocyclic, bicyclic, and polycyclic ring compounds containing from 3 to 20 ring members of which one or more ring member is a heteroatom such as, but not limited to, N, O, and S. Heterocyclic groups include any level of saturation. For instance, heterocyclic groups include unsaturated 3 to 8 membered rings containing 1 to 4 nitrogen atoms; saturated 3 to 8 membered rings containing 1 to 4 nitrogen atoms; condensed unsaturated heterocyclic groups containing 1 to 4 nitrogen atoms; unsaturated 3 to 8 membered rings containing 1 to 2 oxygen atoms and 1 to 3 nitrogen atoms; saturated 3 to 8 membered rings containing 1 to 2 oxygen atoms and 1 to 3 nitrogen atoms; unsaturated condensed heterocyclic groups containing 1 to 2 oxygen atoms and 1 to 3 nitrogen atoms; unsaturated 3 to 8 membered rings containing 1 to 3 sulfur atoms and 1 to 3 nitrogen atoms. Preferred heterocycles contain 5 ring members. Examples of heterocyclic groups include, but are not limited to, 2,4-dihydrofuran. Heterocycles embrace substituted heterocycles or substituted heterocyclic groups according to the definition of “substituted” provided above. The phrase “heterocyclylalkyl” refers to a heterocycle attached through an alkylene moiety. Examples of heterocyclylalkyl include without limitation (pyrrolidine-1-yl)-CH₂—, (piperidine-2-yl)-(CH₂)₂—, and the like.

The term “carboxylate” or “carboxylic acid” refers to COOH; “carboxylic ester” as used herein refers to —COOR²⁷ groups; and the term “amide” (or “amido”) includes C- and N-amide groups, i.e., —C(O)NR²⁸R², and —NR²⁸C(O)R²⁹ groups, respectively. R²⁷ includes is a substituted or unsubstituted alkyl, cycloalkyl, aryl, aralkyl, or heterocyclyl group as defined herein. R²⁸ and R²⁹ are independently hydrogen or R²⁷. Amido groups therefore include but are not limited to carbamoyl groups (—C(O)NH₂) and formamide groups (—NHC(O)H).

II. Preparation of Compounds

Presented below are exemplary general schemes for the preparation of compounds. Further details of synthetic methods are provided in the Examples herein. Since compounds herein can be readily prepared according to procedures well known to one of ordinary skill in the art, numerous methods, in lieu of or in addition to the synthetic schemes presented below, may be employed to prepare compounds herein.

Derivatives and chemically similar compounds within the scope of the instant disclosure may be prepared by routine modification of the procedures provided herein using the appropriate starting materials, the selection of which will be evident to those of skill in the art.

Imidazole-containing compounds presented herein may be prepared according to known methods, using know materials, such as described in U.S. Pat. No. 6,162,804 and J. Med. Chem. 1998, 41:5457, as illustrated by the representative route depicted below in Scheme 1.

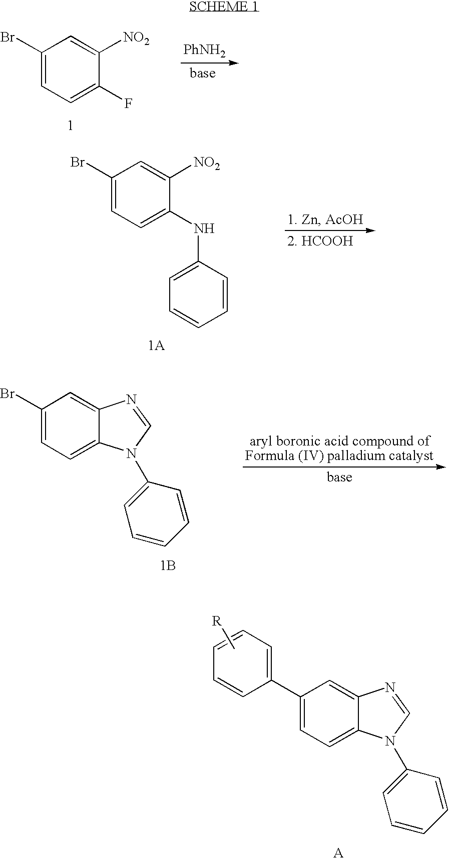

In the exemplary route illustrated above, 1-Bromo-4-fluoro-3-nitrobenzene 1 is combined with aniline followed by addition of a base, such as N,N-diisopropylethylamine, to afford 1A. Conversion of 1A to bromo-benzimidazole 1B is carried out in a two step procedure that involves reduction of the nitro group to an amino group in the presence of zinc and cyclization with formic acid. Aryl boronic acid compounds of Formula (IV) are combined with 1B in the presence of a palladium catalyst to afford exemplary imidazole-containing compounds.

The phrase “palladium catalyst” refers to any carbon-carbon bond forming catalyst comprising palladium. A preferred palladium catalyst for use in preparative methods described herein includes Pd(Ph₃P)₄, PdCl₂(1,1′-Bis(diphenylphosphino)ferrocene).CH₂Cl₂ (i.e., PdCl₂(dppf). CH₂Cl₂), and PdCl₂(Ph₃P)₂.

III. Therapeutic Applications

Compounds and compositions of the instant invention may be used to treat and/or prevent a variety of disorders. “Treating” within the context of the instant invention means an alleviation of symptoms associated with a disorder or disease, or halt of further progression or worsening of those symptoms, or prevention or prophylaxis of the disease or disorder. For example, within the context of cancer, successful treatment may include an alleviation of symptoms or halting the progression of the disease, as measured by a reduction in the growth rate of a tumor, a halt in the growth of the tumor, a reduction in the size of a tumor, partial or complete remission of the cancer, or increased survival rate or clinical benefit. Compounds and compositions that may be used in therapeutic applications have reasonably high bioavailability in a target tissue and acceptably low toxicity. Those skilled in the art can assess compounds described herein for their pharmaceutical acceptability using standard methods.

The phrase “therapeutically effective amount” refers to an amount of a compound described herein sufficient to exert a therapeutically useful effect on the patient treated. The therapeutically effective concentration may be determined empirically by testing the compounds in in vitro and in vivo systems described herein and then extrapolated to determine dosages for humans.

The phrase “effective amount” refers to an amount of a compound described herein sufficient to exert a desired effect. Effective amounts to achieve, for example, inhibition of a particular receptor, such as a tyrosine kinase receptor may be determined empirically by testing the compounds in in vitro and in vivo systems.

For instance, a therapeutically effective amount should produce a serum concentration of active ingredient of from about 0.1 ng/ml to about 50-100 μg/ml. Pharmaceutical compositions should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

Compounds of the instant invention may be used to inhibit, regulate, and/or modulate tyrosine kinase activity, including the signal transduction of tyrosine kinases. In some embodiments, compounds of the invention may be used to inhibit KDR, PDGFR, Kit and FMS. In a preferred embodiment, compounds are used to inhibit or otherwise modulate angiogenesis. Accordingly, compounds of the invention can be utilized to treat and/or prevent indications such as neoangiogenesis, cancer, atherosclerosis, diabetic retinopathy, inflammatory diseases, and the like. Those skilled in the art can determine other diseases and disorders for which administration of compounds or compositions described herein can be beneficial.

The present invention additionally provides prodrugs of the compounds of the invention and pharmaceutically acceptable salts thereof. Prodrugs are compounds which, when metabolized under physiological conditions or when converted by non-physiological conditions, e.g., solvolysis, yield the desired compound. The term “prodrug” contemplates without limitation esters of the active compound wherein during metabolism, the ester group is cleaved to yield the active drug. Further contemplated are prodrugs which are enzymatically activated to yield the active compound, or prodrugs which, upon further chemical reaction, yield the active compound. In this context, an example of a prodrug is an alkyl ester of a carboxylic acid.

Representative Diseases Characterized by Abnormal Cellular Proliferation

In a preferred embodiment, compounds of the present invention may be used in the treatment and/or prevention of abnormal cellular proliferation. Specifically, methods for treating and/or preventing cancer and/or cancerous metastases are contemplated.

1. Epithelial Cancers

For example, a method for treating subjects having a condition characterized by an abnormal epithelial cell proliferation is presented herein. Epithelial cells are cells occurring in one or more layers which cover the entire surface of the body and which line most of the hollow structures of the body, excluding the blood vessels, lymph vessels, and the heart interior which are lined with endothelium, and the chest and abdominal cavities which are lined with mesothelium. Examples of epithelium include anterius corneae, anterior epithelium of cornea, Barrett's epithelium, capsular epithelium, ciliated epithelium, columnar epithelium, epithelium corneae, corneal epithelium, cubical epithelium, cubical epithelium, cuboidal epithelium, epithelium eductus semicircularis, enamel epithelium, false epithelium, germinal epithelium, gingival epithelium, glandular epithelium, glomerular epithelium, laminated epithelium, epithelium of lens, epithelium lentis, mesenchymal epithelium, olfactory epithelium, pavement epithelium, pigmentary epithelium, pigmented epithelium, protective epithelium, pseudostratified epithelium, pyramidal epithelium, respiratory epithelium, rod epithelium, seminiferous epithelium, sense epithelium, sensory epithelium, simple epithelium, squamous epithelium, stratified epithelium, subcapsular epithelium, sulcular epithelium, tessellated epithelium, transitional epithelium.

One category of conditions characterized by abnormal epithelial cell proliferation is proliferative dermatologic disorders. These include conditions such as keloids, seborrheic keratosis, papilloma virus infection (e.g., producing verruca vulbaris, verruca plantaris, verruca plana, condylomata, etc.) and eczema.

An epithelial precancerous skin lesion has a propensity to develop into a cancerous condition. Epithelial precancerous skin lesions also arise from other proliferative skin disorders such as hemangiomas, keloids, eczema and papilloma virus infections producing verruca vulbaris, verruca plantaris and verruca planar. The symptoms of the epithelial precancerous lesions include skin-colored or red-brown macule or papule with dry adherent scales. Actinic keratosis is the most common epithelial precancerous lesion among fair skinned individuals. It is usually present as lesions on the skin which may or may not be visually detectable. The size and shape of the lesions varies. It is a photosensitive disorder and may be aggravated by exposure to sunlight. Bowenoid actinic keratosis is another form of an epithelial precancerous lesion. In some cases, the lesions may develop into an invasive form of squamous cell carcinoma and may pose a significant threat of metastasis. Other types of epithelial precancerous lesions include hypertrophic actinic keratosis, arsenical keratosis, hydrocarbon keratosis, thermal keratosis, radiation keratosis, viral keratosis, Bowen's disease, erythroplaquia of queyrat, oral erythroplaquia, leukoplakia, and intraepidermal epithelialoma.

Another category of conditions characterized by abnormal epithelial cell proliferation is tumors of epithelial origin. Thus, in one aspect, the invention provides a method for treating subjects having epithelial tumors. Epithelial tumors are known to those of ordinary skill in the art and include, but are not limited to, benign and premalignant epithelial tumors, such as breast fibroadenoma and colon adenoma, and malignant epithelial tumors. Malignant epithelial tumors include primary tumors, also referred to as carcinomas, and secondary tumors, also referred to as metastases of epithelial origin. Carcinomas intended for treatment with the methods of the invention include, but are not limited to, acinar carcinoma, acinous carcinoma, alveolar adenocarcinoma (also called adenocystic carcinoma, adenomyoepithelioma, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellularei, glandular carcinoma, granulosa cell carcinoma, hair-matrix carcinoma, hematoid carcinoma, hepatocellular carcinoma (also called hepatoma, malignant hepatoma and hepatocarcinoma), Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypemephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum. In preferred embodiments, the methods of the invention are used to treat subjects having cancer of the breast, cervix, ovary, prostate, lung, colon and rectum, pancreas, stomach or kidney.

2. Sarcomas

Another condition characterized by abnormal cell proliferation to be treated by the methods of the invention include sarcomas. Sarcomas are rare mesenchymal neoplasms that arise in bone and soft tissues. Different types of sarcomas are recognized and these include: liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal [not bone] Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

3. Melanomas

The methods of the invention are also directed towards the treatment of subjects with melanoma. Melanomas are tumors arising from the melanocytic system of the skin and other organs. Examples of melanoma include lentigo maligna melanoma, superficial spreading melanoma, nodular melanoma, and acral lentiginous melanoma.

4. Other Exemplary Cancers

Other conditions characterized by an abnormal mammalian cell proliferation are cancers including, but not limited to, biliary tract cancer, endometrial cancer, esophageal cancer, gastric cancer, intraepithelial neoplasms, including Bowen's disease and Paget's disease, liver cancer, oral cancer, including squamous cell carcinoma, sarcomas, including fibrosarcoma and osteosarcoma, skin cancer, including melanoma, Kaposi's sarcoma, testicular cancer, including germinal tumors (seminoma, non-seminoma (teratomas, choriocarcinomas)), stromal tumors and germ cell tumors, thyroid cancer, including thyroid adenocarcinoma and medullar carcinoma, and renal cancer including adenocarcinoma and Wilms' tumor.

According to other aspects of the invention, methods are provided for treating a subject having an abnormal proliferation originating in blood, bone, muscle or connective tissue. Exemplary conditions intended for treatment by the method of the invention include primary tumors (i.e., sarcomas) of bone and connective tissue.

The methods of the invention are also directed towards the treatment of subjects with metastatic cancers or metastatic tumors. In some embodiments, the metastatic tumors are of epithelial origin. Carcinomas may metastasize to bone, as has been observed with breast cancer, and liver, as is sometimes the case with colon cancer. The methods of the invention are intended to treat metastatic tumors regardless of the site of the metastasis and/or the site of the primary tumor. In preferred embodiments, the metastases are of epithelial origin.

5. Inhibition of Angiogensis

According to a preferred aspect of the invention, methods are provided for inhibiting angiogenesis in disorders having a pathology which requires angiogenesis. Angiogenesis is defined as the formation of new blood vessels. One subset of these disorders is conditions characterized by abnormal mammalian cell proliferation. Another subset is non-cancer conditions including diabetic retinopathy, neovascular glaucoma and psoriasis.

In certain embodiments, the methods of the invention are aimed at inhibiting tumor angiogenesis. Tumor angiogenesis refers to the formation of new blood vessels in the vicinity or within a tumor mass. Solid tumor cancers require angiogenesis particularly for oxygen and nutrient supply. It has been previously shown that inhibition of angiogenesis in solid tumor can cause tumor regression in animal models. Thus in one aspect, the invention relates to a method for inhibiting angiogenesis by inhibiting the proliferation, migration or activation of endothelial cells and fibroblasts, provided this angiogenesis is unrelated to wound healing in response to injury, infection or inflammation.

Thus, in certain embodiments, the methods of the invention are intended for the treatment of diseases and processes that are mediated by angiogenesis including, but not limited to, hemangioma, solid tumors, tumor metastasis, benign tumors, for example hemangiomas, acoustic neuromas, neurofibromas and trachomas, Osler-Webber Syndrome, telangiectasia, myocardial angiogenesis, angiofibroma, plaque neovascularization, coronary collaterals, ischemic limb angiogenesis, corneal diseases, rubiosis, neovascular glaucoma, diabetic retinopathy, retrolental fibroplasia, diabetic neovascularization, macular degeneration, keloids, ovulation, menstruation, and placentation.

IV. Pharmaceutical Compositions

The phrase “pharmaceutically acceptable carrier” refers to any carrier known to those skilled in the art to be suitable for the particular mode of administration. Compounds may optionally be formulated with at least one pharmaceutically acceptable carrier in compositions provided herein.

Compounds described herein may be prepared and/or administered as a pharmaceutically acceptable salt. The phrase “pharmaceutically acceptable salt” refers to any salt preparation that is appropriate for use in a pharmaceutical application. Pharmaceutically-acceptable salts include amine salts, such as salts of N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chloro-benzyl-2-pyrrolidin-1′-ylmethylbenzimidazole, diethylamine and other alkylamines, piperazine, tris(hydroxymethyl)aminomethane, and the like; alkali metal salts, such as lithium, potassium, sodium, and the like; alkali earth metal salts, such as barium, calcium, magnesium, and the like; transition metal salts, such as zinc, aluminum, and the like; other metal salts, such as sodium hydrogen phosphate, disodium phosphate, and the like; mineral acids, such as hydrochlorides, sulfates, and the like; and salts of organic acids, such as acetates, lactates, malates, tartrates, citrates, ascorbates, succinates, butyrates, valerates, fumarates, and the like.

Compositions herein comprise one or more compounds provided herein. The compounds can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Compounds may be formulated into pharmaceutical compositions using techniques and procedures well known in the art. See, e.g., Ansel, INTRODUCTION TO PHARMACEUTICAL DOSAGE FORMS, FOURTH EDITION (1985), 126.

In compositions, one or more compounds is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, or hydrates prior to formulation. The concentrations of the compounds in the compositions are effective for delivery of an amount, upon administration, that treats, prevents, or ameliorates one or more of the symptoms of diseases or disorders to be treated.

Compositions can be formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

V. Therapeutic Administration

Compounds herein may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion, or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art. For example, see REMINGTON'S PHARMACEUTICAL SCIENCES (19^{th} ed.), Mack Publishing Company (Easton, Pa.), 1990.

Dosage forms or compositions containing active compounds in the range of 0.005% to 100% (wt %) with the balance made up from non-toxic carrier may be prepared. Methods for preparation of these compositions are known to those skilled in the art. The contemplated compositions may contain 0.001%-100% (wt %) active compound, in one embodiment 0.1-95% (wt %), in another embodiment 75-85% (wt %).

VI. Combination Therapy

In another embodiment, compounds herein may be administered in combination, or sequentially, with another therapeutic agent. Additionally, compounds of the present invention may be given concurrently with, prior to, or after such treatments. Combination therapy comprising compositions of the present invention may be useful in improving the efficacy of existing therapies for treating certain conditions.

The administration schedule of combination therapy may involve administering the different agents in an alternating fashion. There may be a delay of several hours, days and in some instances weeks between the administration of the different treatments, such that the present compounds may be administered before or after the other treatment. In other embodiments, the agent may be delivered before and during, or during and after, or before and after treatment with other therapeutic agents. In some cases, the present compounds are administered more than 24 hours before the administration of the other treatment agent. In other embodiments, more than one other treatment agent may be administered to a subject. For example, the subject may receive the present compounds, in combination with both surgery and at least one other therapeutic agent. Alternatively, the present compounds may be administered in combination with more than one other therapeutic agent.

In a preferred embodiment, compounds are administered in combination with an anti-cancer compound, such as a cytostatic compound. A cytostatic compound is a compound (e.g., small synthetic molecule, a nucleic acid, a protein) that suppresses cell growth and/or proliferation. In some embodiments, the cytostatic compound is directed towards the malignant cells of a tumor. In yet other embodiments, the cytostatic compound is one which inhibits the growth and/or proliferation of vascular smooth muscle cells or fibroblasts.

Suitable anti-proliferative drugs or cytostatic compounds to be used in combination with the agents of the invention include anti-cancer drugs. Anti-cancer drugs are well known and include: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon α-2a; Interferon α-2b; Interferon α-n1; Interferon α-n3; Interferon β-1a; Interferon γ-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Taxotere; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-cancer drugs include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti cancer compound; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Anti-cancer supplementary potentiating compounds include: tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca²⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and multiple drug resistance reducing compounds such as Cremaphor EL.

Other compounds which are useful in combination therapy for the purpose of the invention include the anti-proliferation compound, Piritrexim Isethionate; the anti-prostatic hypertrophy compound, Sitogluside; the benign prostatic hyperplasia therapy compound, Tamsulosin Hydrochloride; the prostate growth inhibitor, Pentomone; radioactive compounds such as Fibrinogen I¹²⁵, Fludeoxyglucose F¹⁸, Fluorodopa F¹⁸, Insulin I¹²⁵, Insulin I¹³¹, Iobenguane I¹²³, lodipamide Sodium I¹³¹, Iodoantipyrine I¹³¹, Iodocholesterol I¹³¹, Iodohippurate Sodium I¹²³, Iodohippurate Sodium I¹²⁵, Iodohippurate Sodium I¹³¹, Iodopyracet I¹²⁵, Iodopyracet I¹³¹, Iofetamine Hydrochloride I¹²³, Iomethin I¹²⁵, Iomethin I¹³¹, Iothalamate Sodium I¹²⁵, Iothalamate Sodium I¹³¹, Iotyrosine I¹³¹, Liothyronine I¹²⁵, Liothyronine I¹³¹, Merisoprol Acetate Hg¹⁹⁷, Merisoprol Acetate Hg²⁰³, Merisoprol Hg¹⁹⁷, Selenomethionine Se⁷⁵, Technetium Tc 99m Antimony Trisulfide Colloid, Technetium Tc 99m Bicisate, Technetium Tc 99m Disofenin, Technetium Tc 99m Etidronate, Technetium Tc 99m Exametazime, Technetium Tc 99m Furifosmin, Technetium Tc 99m Gluceptate, Technetium Tc 99m Lidofenin, Technetium Tc 99m Mebrofenin, Technetium Tc 99m Medronate, Technetium Tc 99m Medronate Disodium, Technetium Tc 99m Mertiatide, Technetium Tc 99m Oxidronate, Technetium Tc 99m Pentetate, Technetium Tc 99m Pentetate Calcium Trisodium, Technetium Tc 99m Sestamibi, Technetium Tc 99m Siboroxime, Technetium Tc 99m Succimer, Technetium Tc 99m Sulfur Colloid, Technetium Tc 99m Teboroxime, Technetium Tc 99m Tetrofosmin, Technetium Tc 99m Tiatide, Thyroxine I¹²⁵, Thyroxine I¹³¹, Tolpovidone I¹³¹, Triolein I¹²⁵, and Triolein I¹³¹.

Other compounds useful in combination therapies with the inhibitor compounds of the invention include anti-angiogenic compounds such as angiostatin, endostatin, fumagillin, non-glucocorticoid steroids and heparin or heparin fragments and antibodies to one or more angiogenic peptides such as α-FGF, β-FGF, VEGF, IL-8, and GM-CSF. These latter anti-angiogenic compounds may be administered along with the compounds presented herein for the purpose of inhibiting proliferation or inhibiting angiogenesis in all of the aforementioned conditions as described herein. In certain embodiments, the agent may be administered in combination with an anti-angiogenic compound and at least one of the anti-proliferative therapies described above including surgery or anti-proliferative drug therapy.

VII. Kits

According to another aspect of the invention, kits are provided. Kits according to the invention include vessel(s) containing compounds or compositions of the invention.

The phrase “vessel” means any package containing compounds or compositions presented herein. Packaging materials for use in packaging pharmaceutical products are well known to those of skill in the art. See, for example, U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation. In preferred embodiments, the package can be a box or wrapping.

The kit can also contain items that are not contained within the vessel but are attached to the outside of the package, for example, pipettes.

Kits may optionally contain instructions for administering compounds or compositions of the present invention to a subject having a condition in need of treatment. Kits may also comprise instructions for uses of compounds herein approved by regulatory agencies, such as the United States Food and Drug Administration. Kits may optionally contain labeling or product inserts for the present compounds. The package(s) and/or any product insert(s) may themselves be approved by regulatory agencies. The kits can include compounds in the solid phase or in a liquid phase (such as buffers provided) in a package. The kits also can include buffers for preparing solutions for conducting the methods, and pipettes for transferring liquids from one container to another.

The kit may optionally also contain one or more other compounds for use in combination therapies as described herein. In certain embodiments, the package(s) is a container for intravenous administration. In other embodiments, compounds are provided in an inhaler. In still other embodiments compounds are provided in a polymeric matrix or in the form of a liposome.

VIII. Assays to Assess Activity

Various well known in vitro or in vivo assays may be used to evaluate the ability of compounds to modulate tyrosine kinase activity. Additionally, in vitro or in vivo assays may be used to evaluate the ability of compounds to modulate angiogenesis. For example, a two step peptide phosphorylation and detection assay can be used to determine in vitro potencies against a target kinase. In the first stage, a biotinylated substrate peptide containing tandem repeats of poly(glu4-tyr) is incubated with a tyrosine kinase enzyme sample in the presence of non-radioactive ATP and a Mn²⁺/Mg²⁺-co-factor cocktail. In the second step, the phosphorylated substrate is detected by Enzyme Linked Immunosorbent Assay (ELISA) using a monoclonal anti-phosphotyrosine-HRP (Horseradish Peroxidase) antibody conjugate.

The contents of each of the patents, patent applications and journal articles cited in this document are hereby incorporated by reference herein and for all purposes as if fully set forth in their entireties.

The following examples are provided to further illustrate aspects of the invention. These examples are non-limiting and should not be construed as limiting any aspect of the invention.

### EXAMPLES

Unless otherwise noted, materials were obtained from commercial suppliers and used without further purification.

Structural characterization was conducted using ¹H NMR spectroscopy. Proton nuclear magnetic resonance (¹H NMR) spectra were recorded on a Varian AS 500 MHz instrument. Chemical shifts are expressed in ppm downfield from an internal tetramethylsilane standard.

Purified compounds were analyzed for correct mass using a Waters Micromass ZQ mass spectrometer with ESI source. Compound identity was confirmed by the observance of the (M+H⁺) ion (M+1).

### Example 1

### Preparation of Aryl Boronic Acids

Aryl boronic acids were purchased from Frontier Scientific (Logan, Utah) and Acros Organics (Fisher Scientific, Fairlawn, N.J.).

Exemplary aryl boronic acids used in the preparation of compounds of the invention are illustrated below in Table 1.

TABLE 1
Exemplary Aryl Boronic Acids

| Compound | |
|---|---|
| Number | Compound Structure |
| 2 | 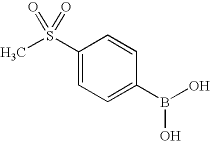 |
| 3 | 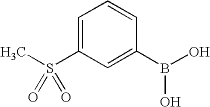 |
| 4 | 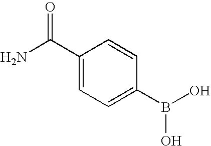 |
| 5 | 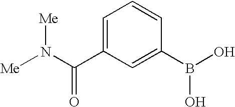 |
| 6 | 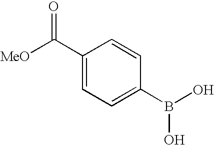 |
| 7 | 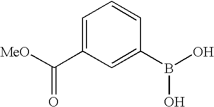 |
| 8 | 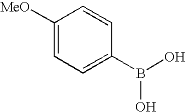 |
| 9 | 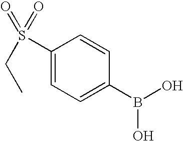 |
| 10 | 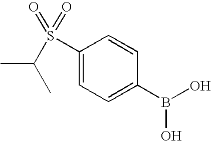 |
| 11 | 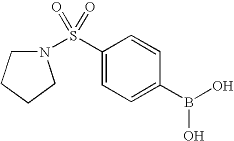 |
| 12 | 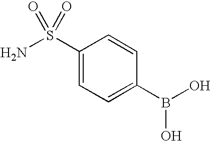 |

### Example 2

### Preparation of Imidazole-Containing Compounds-1

Aryl boronic acid compounds were combined with 1-bromo-4-fluoro-3-nitro-benzene 1 by methods well-known to the art to obtain imidazole-containing compounds of the present invention. As shown in the exemplary route depicted in Scheme 2 and as described below, representative imidazole-containing compounds 13-27 were prepared.

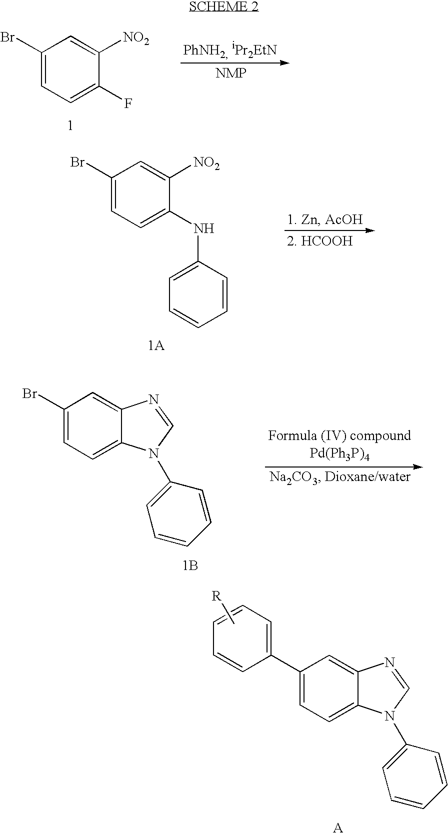

With reference to Scheme 2, base-catalyzed condensation of optionally substituted aniline, or optionally substituted amino heterocycle provides compound 1A, condensation of which in the presence of Zn, AcOH, and HCOOH provides benzimidazole 1B. Reaction of the appropriately substituted boronic acid compound IV in the presence of a palladium catalyst affords exemplary imidazole-containing compounds A.

### Example 3

### Preparation of Imidazole-Containing Compounds-II

Imidazole-containing compounds obtained by the route of Scheme 2 can be further manipulated to introduce various moieties, such as hydrolysis of methyl esters and coupling with 2-piperidin-1-yl-ethylamine or deprotection of methyl ethers, followed by alkylation with 2-chloro-N-(2-piperidin-1-yl-ethyl)-acetamide. For example, Scheme 3 provides a synthetic pathway for the piperidyl-ethylamide 17 compound of the invention from methyl ester 17A.

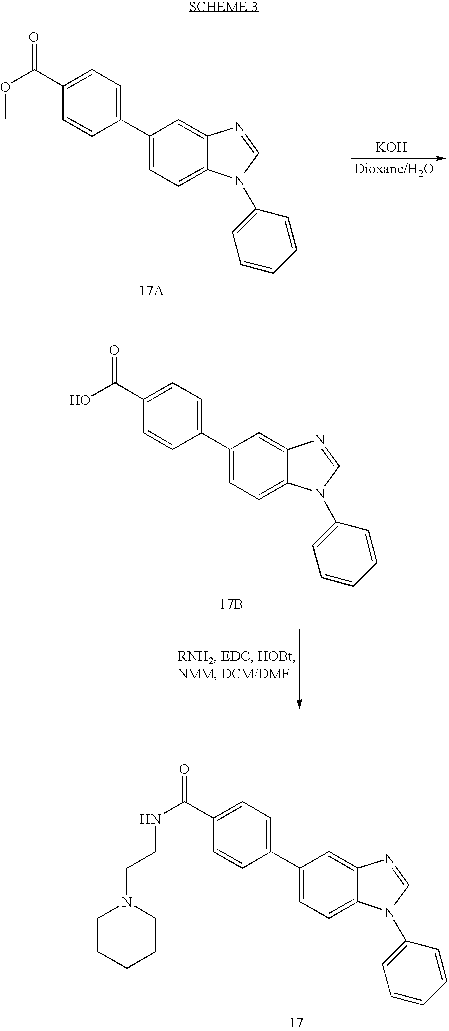

### Step 1—Preparation of 4-(1-Phenyl-1H-benzoimidazol-5-yl)-benzoic acid (17B)

To a solution of 4-(1-Phenyl-1H-benzoimidazol-5-yl)-benzoic acid methyl ester (Cmpd 13A, 124 mg, 0.38 mmol) in dioxane (5 mL) was added a 2N KOH aqueous solution (1 mL) dropwise. The reaction mixture was vigorously stirred for 40 hours. Solvents were then removed under reduced pressure. The solid residue was purified by silica gel column chromatography to yield 17B as a white solid (76 mg, 64% yield). ¹H-NMR (500 MHz, DMSO) δ 8.63 (s, 1H), 8.11 (m, 1H), 8.02 (d, J=8.3 Hz, 2H), 7.78 (d, J=8.3 Hz, 2H), 7.73 (m, 4H), 7.66 (m, 2H), 7.53 (m, 1H). MS m/z (rel intensity) 315 (M+1) (100).

### Step 2—Preparation of 4-(1-Phenyl-1H-benzoimidazol-5-yl)-N-(2-piperidin-1-yl-ethyl)-benzamide (Cmpd 17)

To a solution of acid 17B (154.5 mg, 0.5 mmol) in CH₂Cl₂ (6 mL) and DMF (2 mL) cooled to 0° C. in an ice bath was added HOBt (75 mg, 0.5 mmol). After 10 minutes, EDC (113 mg, 0.6 mmol) was added. After 15 minutes, 2-piperidin-1-yl-ethylamine (78 μL, 0.5 mmol) and NMM (108 μL, 1 mmol) were sequentially added. The reaction solution was allowed to warm up to room temperature and stirred overnight. The reaction mixture was diluted with additional CH₂Cl₂ (5 mL) and washed with NaHCO₃ (2×10 mL). The organic layer was dried over Na₂SO₄ and evaporated under reduced pressure. The resulting oily residue was purified by silica gel column chromatography and recrystallization from DCM/hexanes to afford compound 17 (93 mg, 45% yield). ¹H-NMR (500 MHz, CDCl₃) δ 8.17 (s, 1H), 8.12 (s, 1H), 8.01 (d, J=8.3 Hz, 2H), 7.76 (d, J=8.3 Hz, 2H), 7.61 (m, 4H), 7.55 (m, 2H), 7.50 (dd, J=7.3, 7.3 Hz, 1H), 3.70 (m, 2H), 2.76 (m, 6H), 1.80 (m, 4H), 1.56 (m, 2H). MS m/z (rel intensity) 425 (M+1) (42), 340 (26), 213 (28), 171 (100).

Illustrated below in Tables 2 and 3 are exemplary benzimidazole compounds which were obtained using the representative preparative routes illustrated in Schemes 2 and 3.

TABLE 2
Exemplary Compounds of the Invention

| | | | MS |
|---|---|---|---|
| Compound | | | [m/z] |
| Number | Compound Structure | ¹H NMR | (rel intensity) |
| 13 | 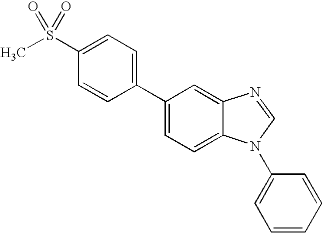 | -at 500 MHz in CDCl₃δ8.19 (s, 1H) δ8.12 (m, 1H) δ8.03 (d, J=8.3 Hz, 2H) δ7.85 (d, J=8.3 Hz, 2H) δ7.62 (m, 4H) δ7.53 (m, 3H) δ3.11 (s, 3H) | [M + 1] = 349 (100) |
| 14 | 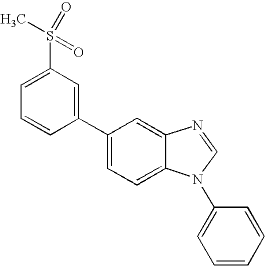 | -at 500 MHz in CDCl₃δ8.24 (dd, J=1.5, 1.5 Hz, 1H) δ8.19 (s, 1H) δ8.11 (m, 1H) δ7.94 (m, 2H) δ7.64 (m, 5H) δ7.54 (m, 3H) δ3.13 (s, 3H) | [M + 1] = 349 (100) |
| 15 | 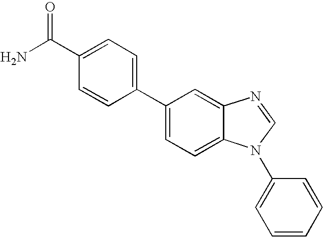 | -at 500 MHz in DMSO-d6 δ8.64 (s, 1H) δ8.14 (s, 1H) δ8.02 (bs, 1H) δ7.98 (d, J=8.5 Hz, 2H) δ7.83 (d, J=8.5 Hz, 2H) δ7.73 (m, 4H) δ7.66 (dd, J=7.5, 7.5 Hz, 2H) δ7.53 (dd, J=7.3, 7.3 Hz, 1H) δ7.37 (bs, 1H) | [M + 1] = 314 (100) |
| 16 | 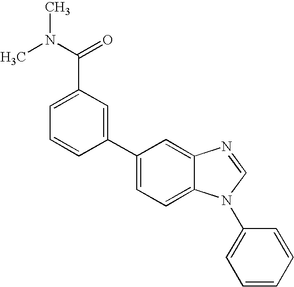 | -at 500 MHz in CDCl₃δ8.16 (s, 1H) δ8.06 (m, 1H) δ7.71 (m, 2H) δ7.61 (m, 4H) δ7.56 (m, 2H) δ7.50 (m, 2H) δ7.40 (m, 1H) δ3.16 (bs, 3H) δ3.06 (bs, 3H) | [M + 1] = 342 (100) |
| 17 | 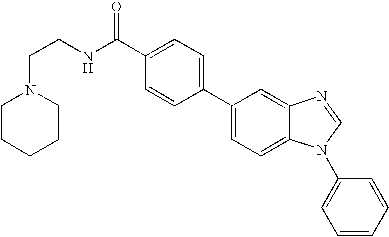 | -at 500 MHz in CDCl₃δ8.17 (s, 1H) δ8.12 (s, 1H) δ8.01 (d, J=8.3 Hz, 2H) δ7.76 (d, J=8.3 Hz, 2H) δ7.61 (m, 4H) δ7.55 (m, 2H) δ7.50 (dd, J=7.3, 7.3 Hz, 1H) δ3.70 (m, 2H) δ2.76 (m, 6H) δ1.80 (m, 4H) δ1.56 (m, 2H) | [M + 1] = 425 (42) |
| 18 | 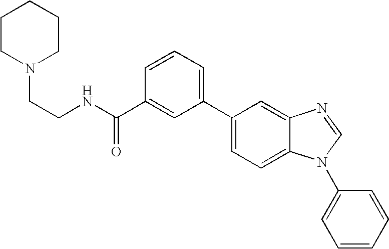 | -at 500 MHz in CD3OD δ8.51 (s, 1H) δ8.22 (m, 1H) δ8.06 (s, 1H) δ7.93 (d, J=7.8 Hz, 1H) δ7.87 (d, J=7.8 Hz, 1H) δ7.72 (bs, 2H) δ7.68 (m, 4H) δ7.61 (dd, J=7.5, 7.5 Hz, 1H) δ7.56 (m, 1H) δ3.80 (d, J=6.0 Hz, 2H) δ3.34 (m, 6H) δ1.89 (m, 6H) | [M + 1] = 425 (18) |

TABLE 3
Further Exemplary Compounds of the Invention

| | | MS |
|---|---|---|
| Compound | | [m/z] |
| Number | Compound Structure | (rel intensity) |
| 19 | 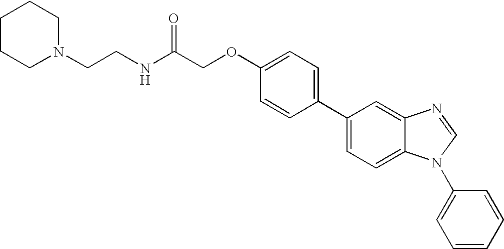 | [M + 1] = 455 (100) |
| 20 | 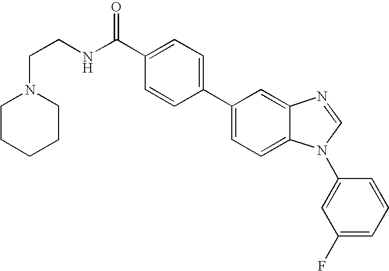 | [M + 1] = 443 (31) |
| 21 | 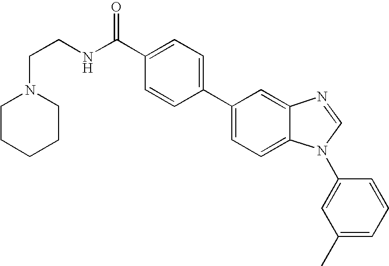 | [M + 1] = 439 (5) |
| 22 | 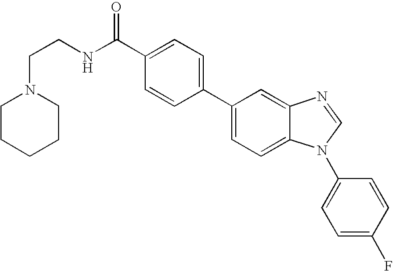 | [M + 1] = 443 (18) |
| 23 | 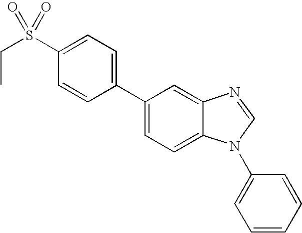 | [M + 1] = 363 (100) |
| 24 | 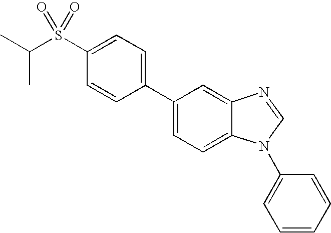 | [M + 1] = 377 (100) |
| 25 | 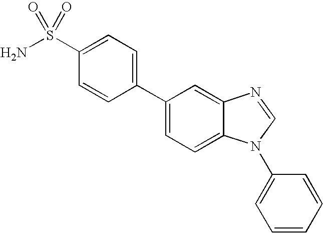 | [M + 1] = 350 (100) |
| 26 | 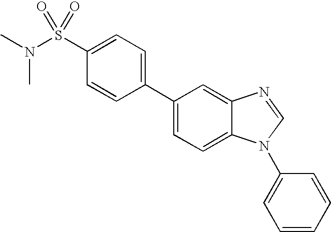 | [M + 1] = 378 (100) |
| 27 | 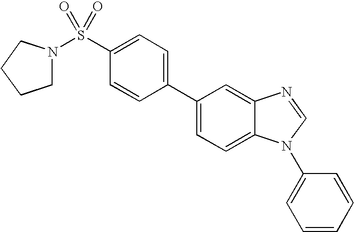 | [M + 1] = 404 (100) |

### Example 4

### Tyrosine Kinase Assay

A direct ELISA assay for the chemiluminescent detection of protein tyrosine phosphotransferase activity using a monoclonal anti-phosphotyrosine-HRP conjugate detection antibody can be used to measure kinase activity of PDGFRβ. A biotinylated substrate peptide containing tandem repeats of Poly (Glu₄-Tyr) is incubated with PDGFRβ, in the presence of 5 μM ATP and a Mn²⁺/Mg²⁺ co-factor cocktail. The phosphorylated substrate is detected by direct Enzyme Linked Immunosorbent Assay (ELISA) using a monoclonal anti-phosphotyrosine-HRP (Horseradish Peroxidase) antibody conjugate. A chemiluminescent substrate is used to detect the HRP conjugate.

A detailed protocol for IC₅₀ determination is as follows: 96 well plates are first coated overnight with an excess neutravidin biotin binding protein and then are washed in a plate washer (Tecan Power Washer 384) three times with TBS-Tween to remove unbound protein. Biotinylated peptide substrate (Poly(Glu₄-Tyr) Peptide, Upstate, catalog #12-440) at concentration of 1 μg/ml is incubated for 90 minutes to allow sufficient capture of the substrate by the neutravidin coating. Compounds at various concentrations (half log dilution for 8 dose assay or log dilutions for 3 dose assays) are incubated with PDGFRβ (Upstate) at the final concentration of 500 ng/ml in a kinase buffer containing 60 mM Hepes pH 7.5, 3 mM MgCl₂, 3 mM MnCl₂, 150 uM EGTA, 15 μM Sodium Orthovanadate, 1 mM DTT, 500 μg/mL BSA, 20 μg/ml PEG, and 5 μM ATP. The reaction was allowed to proceed for 20 minutes before washing the plate. The degree of phosphorylation of the peptide is measured using an HRP conjugated anti-phosphotyrosine antibody (Anti-phosphotyrosine, 4G10, Upstate, catalog #16-105) and is detected with a chemiluminescent substrate. IC₅₀ and IC₉₀ calculations are performed by non-linear regression analysis using Prism software (GraphPad).

Compounds of the invention may be tested according to the above assay. They have been or will be found to inhibit PDGFRβ.

What is claimed is:

1. A compound corresponding to Formula (I), or a pharmaceutically acceptable salt, hydrate, or prodrug thereof: 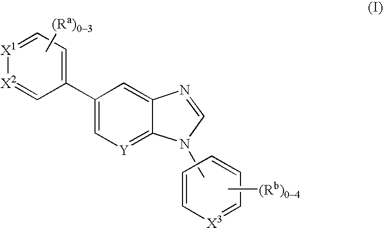
wherein:
each of X¹ and X² is independently CH, CR^{a}, or N, provided that at least one of X¹ or X² is CR¹;
X³ is CH, CR^{b}, or N;
Y is CH or N;
R^{a} at each occurrence is independently alkenyl, alkynyl, OR¹, C(O)NR²R³, C(O)NR²(C₁-C₆ alkylene-R¹), C(O)OR², O—(C₁-C₆ alkylene)-C(O)NR²R³, O—(C₁-C₆ alkylene)-C(O)NR²(C₁-C₆ alkylene-R¹), SR², SO₂R², SO₂NR²R³, NR²SO₂R³, SO₃R², O—(C₁-C₆ alkylene)-NR²C(O)R³, O—(C₁-C₆ alkylene)-NR²C(O)(C₁-C₆ alkylene-R¹), NR²—(C₁-C₆ alkylene)-C(O)NR²R³; or NR²—(C₁-C₆ alkylene)-C(O)NR²(C₁-C₆ alkylene-R¹);
R^{b} is optional, and when present at each occurrence is independently R², halogen, alkyl, alkenyl, alkynyl, alkoxy, C₁-C₆ alkylene-R¹, NH(C₁-C₆ alkylene-R¹), OR¹, OR², NR²R³, NO₂, C(O)NR²R³, C(O)OR², O—(C₁-C₆ alkylene)-C(O)NR²R³, SR², SO₂R², SO₂NR²R³, NR²SO₂R³, SO₃R², O—(C₁-C₆ alkylene)-NR²(O)R³, or N—(C₁-C₆ alkylene)-C(O)NR²R³;
R¹ at each occurrence is independently selected from aryl, heteroaryl, or heterocyclyl; and
R² and R³ are at each occurrence independently selected from hydrogen, alkyl, alkenyl, alkynyl, alkoxy, aryl, heteroaryl, cycloalkyl, cycloalkylalkyl, heterocyclyl, or heterocyclylalkyl; or R² and R³, are taken together, to form a cycloalkyl or heterocyclyl.

2. The compound of claim 1, corresponding to Formula (II):

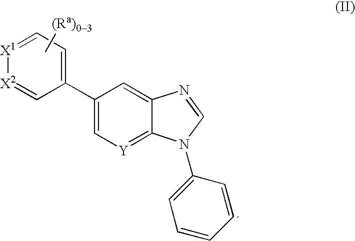

3. The compound of claim 2, corresponding to Formula (III):

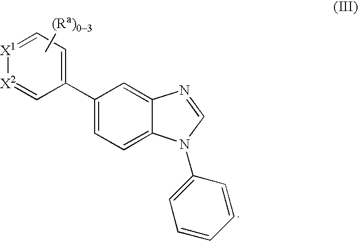

4. The compound of claim 3, wherein X¹ is CR^{a} and X² is CH.

5. The compound of claim 4, wherein R^{a} is SO₂(alkyl), C(O)NH₂, C(O)NH—(C₁-C₆ alkylene)-heterocyclyl, SO₂NR²R³, or O—(C₁-C₆ alkylene)-C(O)NH—(C—C₆ alkylene)-heterocyclyl.

6. The compound of claim 5 selected from the group consisting of:

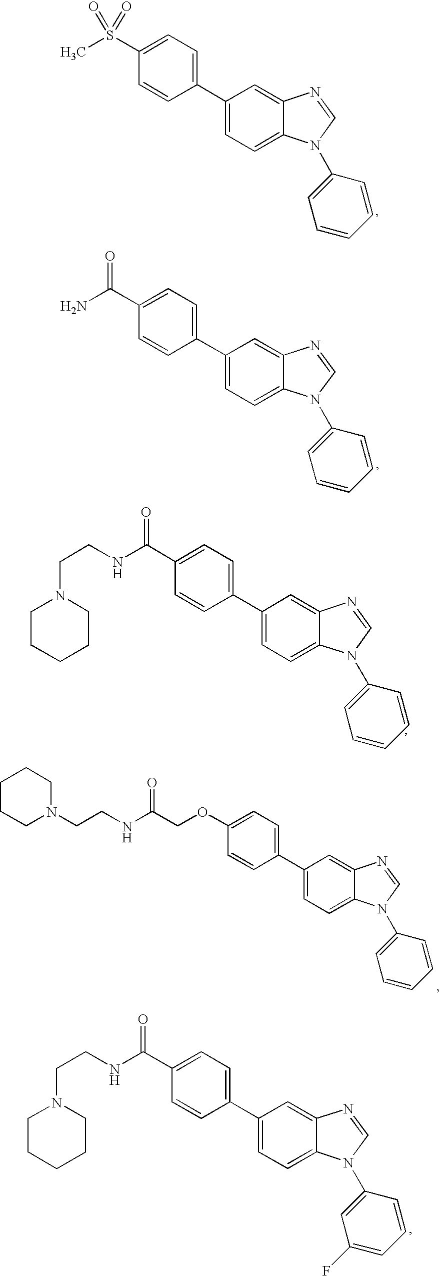

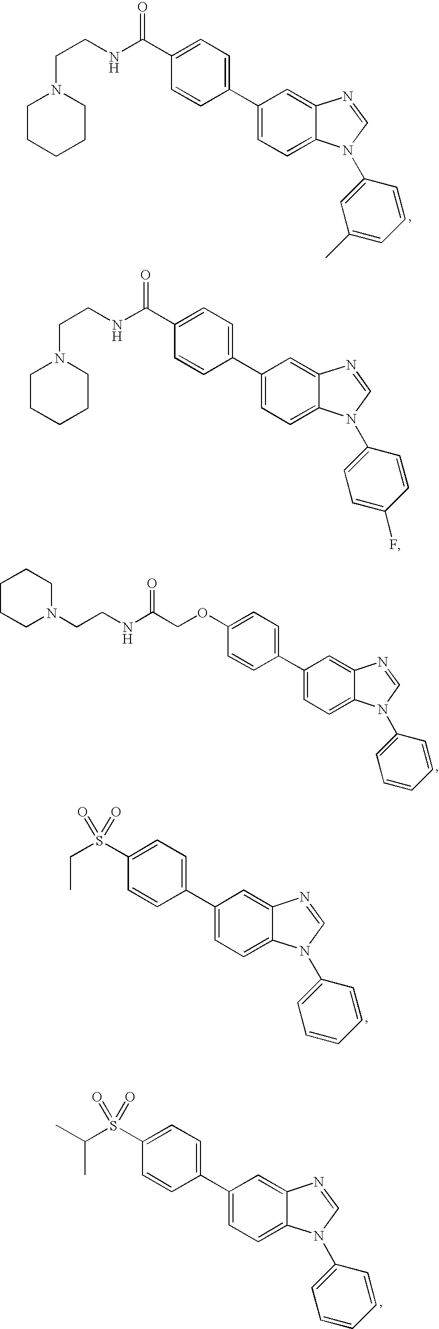

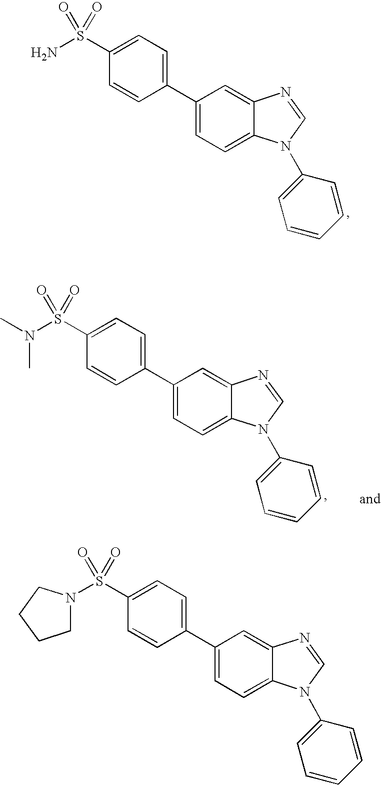

7. The compound of claim 3, wherein X¹ is CH and X² is CR^{a}.

8. The compound of claim 7 wherein R^{a} is C(O)NR²R³ or SO₂R².

9. The compound of claim 8 selected from the group consisting of

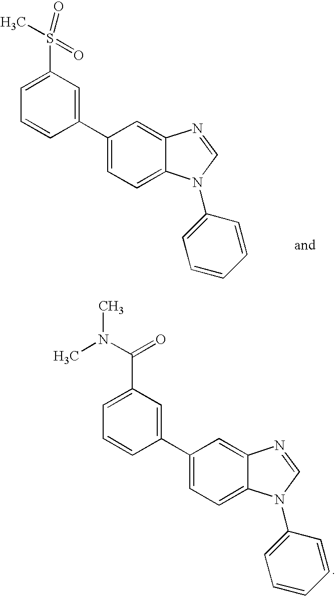

10. The compound of claim 7, wherein R^{a} is C(O)NH—(C₁-C₆ alkylene)-heterocyclyl.

11. The compound of claim 10 that is:

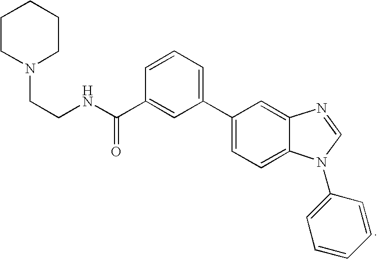

12. A composition comprising the compound of claim 1 and a pharmaceutically acceptable carrier.

13. A method of modulating tyrosine kinase activity comprising administering an effective amount of the compound of claim 1, whereby activity of said tyrosine kinase is altered.

14. A method of modulating angiogenesis comprising administering an effective amount of the compound of claim 1, whereby said compound modulates a tyrosine kinase involved in angiogenesis.

15. A method of treating a disease characterized by abnormal cellular proliferation comprising administering a therapeutically effective amount of the compound of claim 1 to a patient in need thereof.

16. The method of claim 15, wherein said disease is cancer and is selected from cancers of the brain, genitourinary tract, lymphatic system, stomach, larynx, or lung.

17. A method of treating an inflammatory disease comprising administering a therapeutically effective amount of the compound of claim 1 to a patient in need thereof.

18. The method of claim 17, wherein said inflammatory disease is selected from rheumatoid arthritis, psoriasis, contact dermatitis, or delayed hypersensitivity reactions.

19. A method for preparing the compound of claim 1, said method comprising combining compound corresponding to formula (IV): 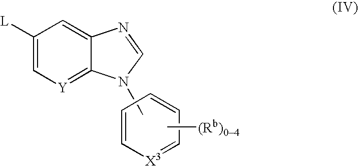
with a compound corresponding to Formula (V) in the presence of a palladium catalyst, wherein said compound of Formula (V) corresponds to: 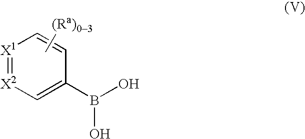
wherein:
L is a leaving group;
each of X¹ and X² is independently CH, CR^{a}, or N, provided that at least one of X¹ or X² is CR^{a};
X³ is CH, CR^{b}, or N;
Y is CH or N;
R^{a} at each occurrence is independently alkenyl, alkynyl, OR¹, C(O)NR²R³, C(O)NR²(C₁-C₆ alkylene-R¹), C(O)OR², O—(C₁-C₆ alkylene)-C(O)NR²R³, O—(C₁-C₆ alkylene)-C(O)NR²(C₁-C₆ alkylene-R¹), SR², SO₂R², SO₂NR²R³, NR²SO₂R³, SO₃R², O—(C₁-C₆ alkylene)-NR²C(O)R³, O—(C₁-C₆ alkylene)-NR²C(O)(C₁-C₆ alkylene-R¹), NR²—(C₁-C₆ alkylene)-C(O)NR²R³; or NR²—(C₁-C₆ alkylene)-C(O)NR²(C₁-C₆ alkylene-R¹);
R^{b} is optional, and when present at each occurrence is independently R², halogen, alkyl, alkenyl, alkynyl, alkoxy, C₁-C₆ alkylene-R¹, NH(C₁-C₆ alkylene-R¹), OR¹, OR², NR²R³, NO₂, C(O)NR²R³, C(O)OR², O—(C₁-C₆ alkylene)-C(O)NR²R³, SR², SO₂R², SO₂NR²R³, NR²SO₂R³, SO₃R², O—(C₁-C₆ alkylene)-NR²C(O)R³, or N—(C₁-C₆ alkylene)-C(O)NR²R³;
R¹ at each occurrence is independently selected from aryl, heteroaryl, or heterocyclyl; and
R² and R³ are at each occurrence independently selected from hydrogen, alkyl, alkenyl, alkynyl, alkoxy, aryl, heteroaryl, cycloalkyl, cycloalkylalkyl, heterocyclyl, or heterocyclylalkyl; or R² and R³, are taken together, to form a cycloalkyl or heterocyclyl.

20. The method of claim 19, wherein L is Br, I, or triflate.
